(19) United States
(12) Patent Application Publication
(10) Pub. No.: US 2019/0293096 A1
(43) Pub. Date: Sep. 26, 2019

(54) MEDICAL LINE MANAGEMENT DEVICE

(71) Applicant: The National College of Art and Design, Dublin (IE)

(72) Inventor: Eoin Ormonde, Dublin (IE)

(73) Assignee: The National College of Art and Design, Dublin (IE)

(21) Appl. No.: 16/362,368

(22) Filed: Mar. 22, 2019

Related U.S. Application Data
(60) Provisional application No. 62/647,474, filed on Mar. 23, 2018.

(51) Int. Cl. F16B 2/10; F16B 2/00; F16B 2/24

(52) U.S. Cl. CPC F16B 2/10; A61M 2025/024; F16B 2/245; F16B 2/005

(57) ABSTRACT

Disclosed is a medical line management device for organisation and/or arrangement of tubes, hoses, catheters and wires for example in the environment of a hospital bed. In one embodiment, disclosed is a grip for guiding at least one wire that includes at least one clamp adapted to reversibly fasten the grip to a surface, and at least one guide adapted to receive the one or more wires, wherein the guide includes at least one aperture.

### CROSS REFERENCE TO RELATED APPLICATION(S)

This claims the benefit of U.S. Provisional Application No. 62/647,474, filed Mar. 23, 2018, incorporated herein by reference in its entirety.

### FIELD

The present invention is in the field of medical appliances, more specifically devices for use in the care of a patient confined to a hospital bed, more specifically the organisation and/or arrangement of tubes, hoses, catheters and wires for example in the environment of a hospital bed.

### BACKGROUND

A hospital is a busy system of machines and devices that need to be moved and positioned constantly by nurses. The majority of these devices have electrical wires or medical tubing or hoses or catheters attached. Due to the number of wires, tubes, hoses, and/or catheters coming into close contact with each other, entanglement can often occur.

This can result in a number of problems.

Firstly, such entanglement can be a hazard to patients and hospital staff, who could trip as a result of loose wires, tubes, hoses, and/or catheters, particularly patients who may be elderly or infirm, and susceptible to falls.

Secondly, Nurses and other hospital staff have to spend valuable time untangling the wires, tubes, hoses, and/or catheters; as the case may be.

A patient can be connected to many wires, tubes, hoses, and/or catheters at any one time, for example it is not uncommon for up to seven IV tubes to be running off a hospital bed along with additional monitoring wires. For example in the intensive care unit this may be accepted as part of the setup.

In particular, it can be difficult to label and/or identify a particular wire, tube, hose, and/or catheter when several wires, tubes, hoses, and/or catheters are on top of each other.

Additional problems can arise, when patient medications are changed or a patient has to be moved to a new setting, for example, when a patient in an intensive care unit needs to be brought into an operating theatre. In this situation, medical devices must be disconnected and reconnected. Patients are routinely connected to medical devices with wires, tubes, hoses, and/or catheters, for example for the purpose of delivering medications, gases and/or food. In this context, medical devices having wires, tubes, hoses, and/or catheters cause particular problems as said wires, tubes, hoses, and/or catheters have to be attached and re-attached. Such medical devices frequently have similar and often identical connectors. The related wires, tubes, hoses, and/or catheters also frequently have similar and often identical connectors. The multitude of similar connectors in a clinical environment can often lead to difficulties in identifying the correct wires, tubes, hoses, and/or catheters, which can result in a clinician misconnecting two devices which have different intended purposes. Such misconnection can have dire clinical consequences. Indeed, the clinician often has to trace all lines (for example wires, tubes, hoses, and/or catheters) back to their origin before connecting or disconnecting any devices or infusions. This task is time-consuming and requires concentration as the nurse or doctor must first identify each line, tracing each back to its origin and detaching and reattaching to the new system or medical device. Time and mental effort on the part of the clinician is also consumed in line reconciliation: that is rechecking connections and tracing all patient wires, tubes, hoses, and/or catheters to their sources upon the patient's arrival to a new setting or service as part of the hand-off process.

The mobility of wires, tubes, hoses, and/or catheters and related equipment is also an issue in a hospital setting. Frequently, when moving a patient who is connected to at least one line (such as one or more wires, tubes, hoses, and/or catheters) the lines may become caught.

Any wires, tubes, hoses, and/or catheters that are on the floor, partly on the floor, trailing on the floor, or hanging down toward the floor, can become caught when moving machinery, equipment, medical devices or beds; and also represent a trip hazard for patients, clinicians and other staff and visitors.

Additional difficulties may arise when trying to dress or undress patients who are connected to lines (such as an IV line, or other wires, tubes, hoses, and/or catheters), where the line can become caught for example on the patient's clothing, on the hospital bed, on an assisting clinicians clothing.

A lack of space is a common problem in clinical settings. The lack of space frequently affects the placement of machines and equipment. As a consequence, hospital beds and machinery may be stored in corridors, which may result in any wires, tubes, hoses, and/or catheters relating to a first hospital bed being brought into closer contact with any wires, tubes, hoses, and/or catheters relating to one or more other hospital beds, and this can exacerbate the problems described above.

Nurse call devices are integral for a patient to conveniently call a nurse for aid, especially where the patient is bed-bound. The nurse-call device must be visible to the patient and within reach, especially for elderly or infirm patients who may need access to the device at all times when in a hospital bed. A nurse call device is usually connected to equipment near the patient's hospital bed by wire. The wire may be for example 8 foot long. This long wire risks entanglement with other wires, tubes, hoses, and/or catheters.

One problem occurs when patients accidentally knock or push the nurse call device to the floor, in which case the nurse call device is no longer accessible and the patient has to physically call the nurse. This may mean that the patient remains uncomfortable or does not receive the appropriate care or treatment. There is no prevention method in place to stop this happening except the existing pillowclip which merely guides the wire and does very little to constrain the device to prevent falling.

Solution

After researching clip and hinge mechanisms I found a way to give a simple inverted clip function by halving and slotting a hinge.

The grip of the present invention provides a simple and neat solution for clamping the bed sheet.

One challenge was finding a solution in order for the grip of the present invention to lock around, or guide wires, tubes, hoses, and/or catheters of multiple diameters. Ideally, a clinician's interaction with the grip of the present invention to lock and unlock such wires, tubes, hoses, and/or catheters should be one handed, as one hand is unlocking the grip the other hand will be moving the wires, tubes, hoses, and/or catheters into the unlock position.

A general problem which this invention solves is the problem of how to make the identification of wires and tubes easier in a hospital or hospital bed environment, in order to make the task of attachment and reattachment easier and more efficient, to give a quicker and streamlined identification and attachment task without disturbing the function of any wires movement or accessibility. Also, how to reduce medical entanglement for hospital environments.

The present invention controls lines (for example wires, tubes, hoses, and/or catheters) entanglement, by constraining and organising such lines.

The present invention for the first time allows for clean, simple and efficient line tracing.

The solution of the present invention is unobtrusive.

The solution of the present invention does not disrupt the function of the lines (for example wires, tubes, hoses, and/or catheters), their movement or accessibility.

Materials

It would be advantageous if the grip of the present invention were manufactured of materials that are resistant to sterilisation chemicals. It would be advantageous if the grip of the present invention were manufactured of materials that are antibacterial.

### SUMMARY

Disclosed is a medical line management device for organisation and/or arrangement of tubes, hoses, catheters and wires for example in the environment of a hospital bed. In one embodiment, disclosed is a grip for guiding at least one wire that includes at least one clamp adapted to reversibly fasten the grip to a surface, and at least one guide adapted to receive the one or more wires, wherein the guide includes at least one aperture.

### BRIEF DESCRIPTION OF THE FIGURES

The invention will now be described in detail with reference to specific embodiments and with reference to the accompanying drawings, in which:

FIG. 1 shows a line-drawing side view of a first embodiment of a grip in accordance with the invention, wherein the clamp is in the closed position.

FIG. 2 shows a line-drawing perspective view of a first embodiment of a grip in accordance with the invention, wherein the clamp is in the open position.

FIG. 3 shows a line-drawing perspective view of a first embodiment of a grip in accordance with the invention, wherein the clamp is in the closed position.

FIG. 4 shows a computer-generated perspective view of a second embodiment of a grip in accordance with the invention.

FIG. 5 shows a photograph of a second embodiment of a grip in accordance with the invention.

FIG. 6 shows a photograph of a third embodiment of a grip in accordance with the invention.

FIG. 7 shows a line-drawing side view of a third embodiment of a grip in accordance with the invention, wherein the clamp is in the closed position.

FIG. 8 shows a line-drawing perspective view of a third embodiment of a grip in accordance with the invention, wherein the clamp is in the closed position.

FIG. 9 shows a line-drawing perspective view of a third embodiment of a grip in accordance with the invention, wherein the clamp is in the open position.

FIG. 10 shows a line-drawing side view of a third embodiment of a grip in accordance with the invention.

FIG. 11 shows a photograph of a fourth embodiment of a grip in accordance with the invention.

FIG. 12 shows a computer generated perspective view of a fifth embodiment of a grip in accordance with the invention.

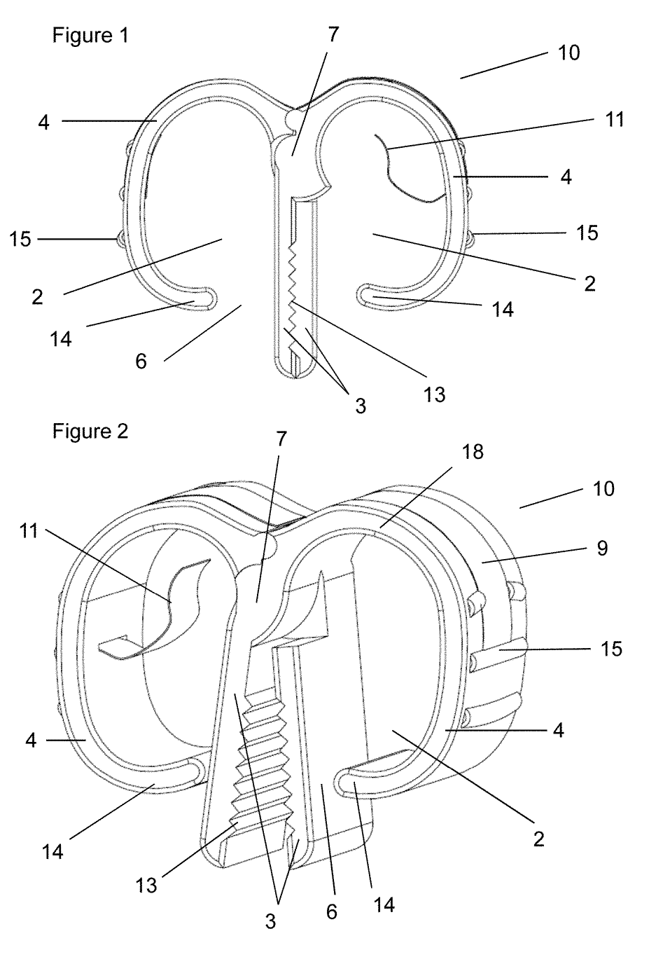

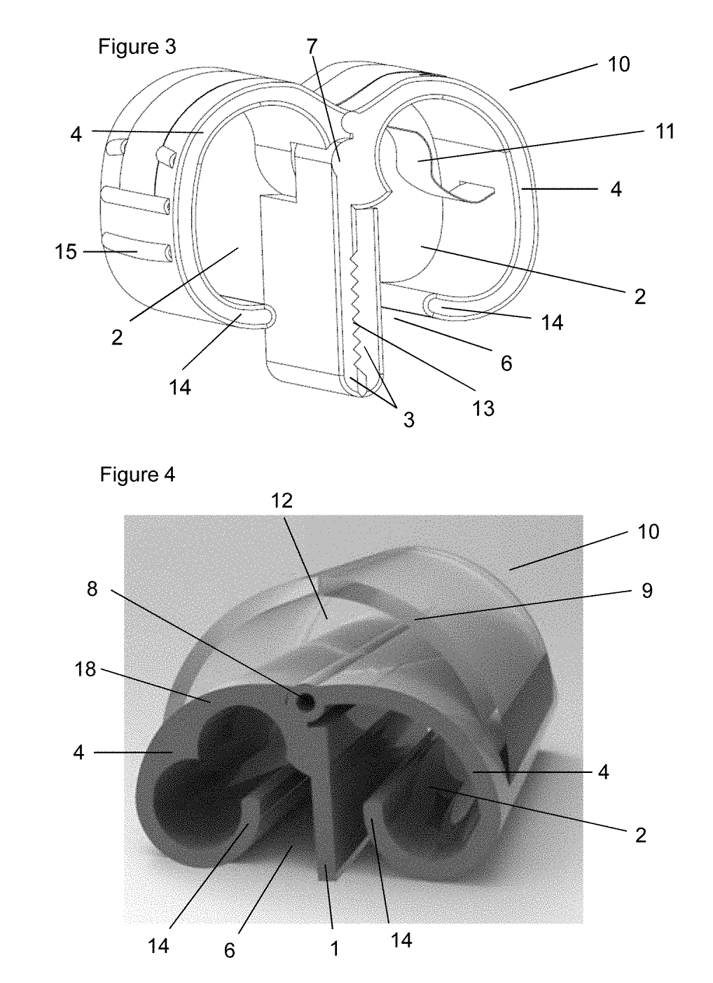

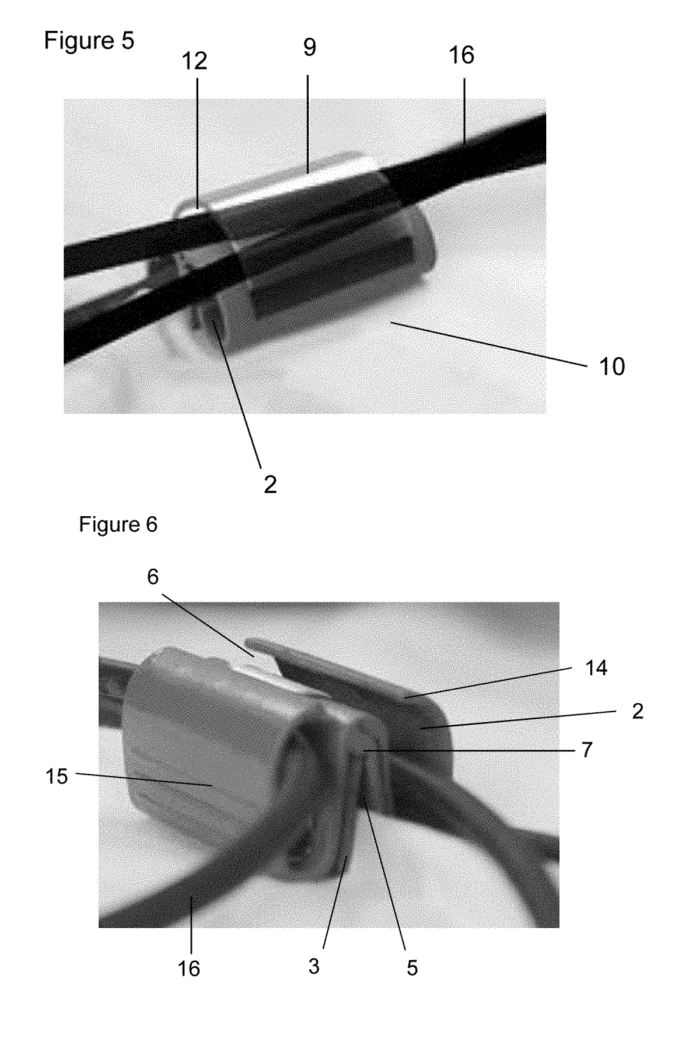

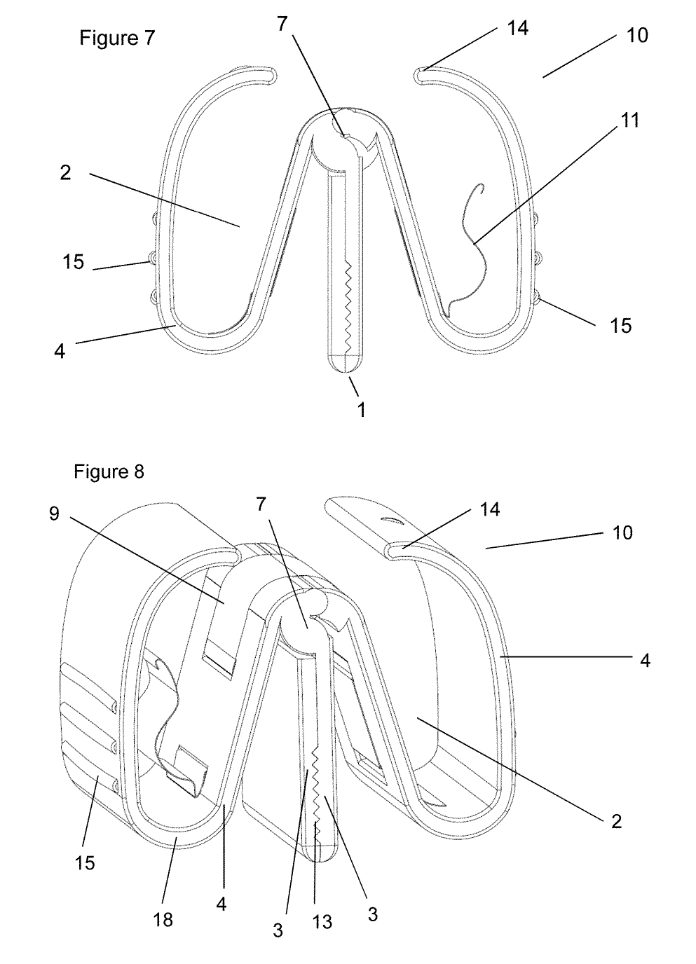

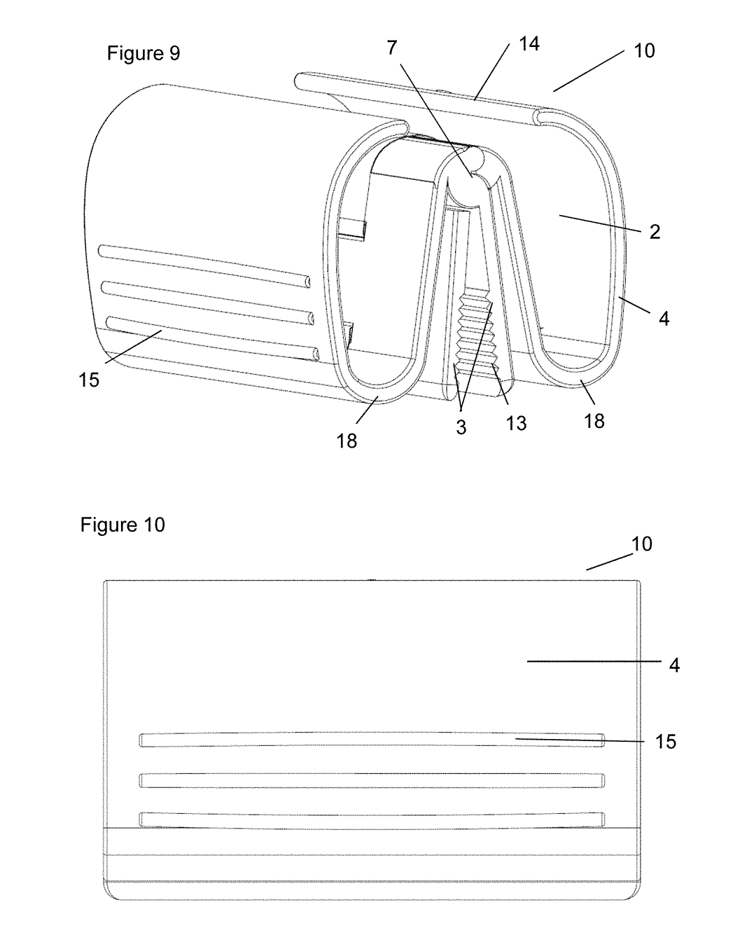

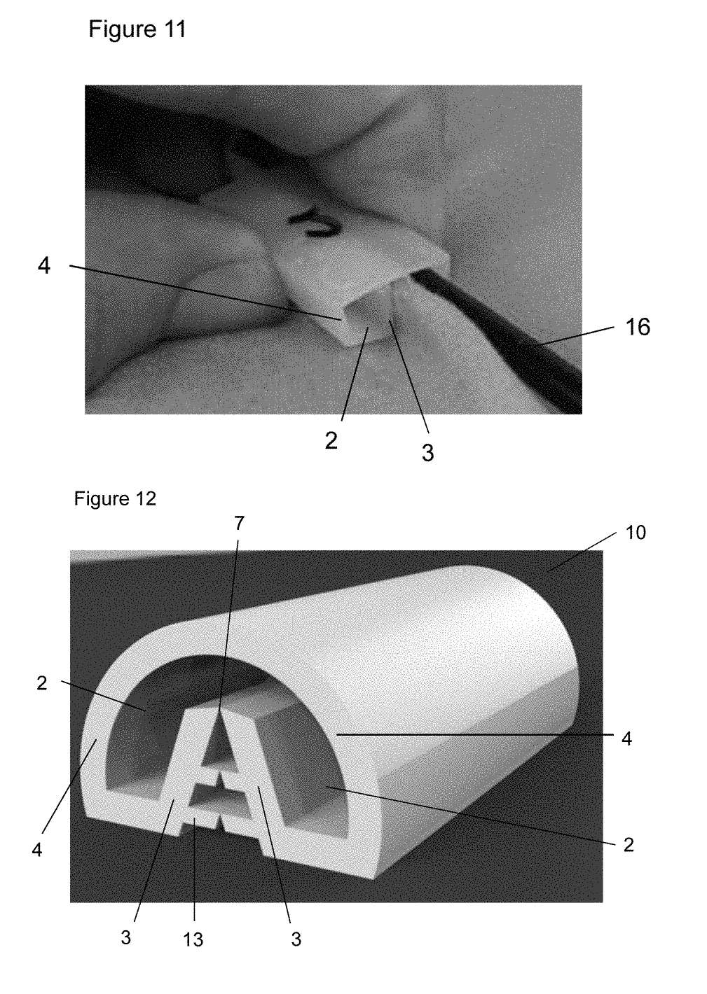

### DETAILED DESCRIPTION

In some embodiments, there is provided a grip for guiding one or more wires, the grip comprising:
(a) at least one clamp adapted to reversibly fasten the grip to a surface, and
(b) at least one guide adapted to receive the one or more wires.

Optionally, the guide comprises at least one aperture.

Optionally, the grip comprises at least one lip adjacent to the aperture wherein the at least one lip is formed from resilient material.

Optionally, the grip is a unitary grip.

Optionally, the grip is formed as a single continuous part.

Optionally, the grip is formed from one or more parts.

Optionally, the grip is formed from a single material.

Optionally, the grip is formed from two or more materials. The materials can be the same or different materials.

Optionally, the grip is formed from a resilient material.

Optionally, the grip is formed from a single resilient material.

Optionally, the grip is formed from two or more resilient materials. The materials can be the same or different resilient materials.

Optionally, the grip comprises at least one material selected from the group comprising: plastic, rubber, synthetic resin, glass fibre, and metal.

Optionally, the grip comprises plastic. Optionally, the grip comprises a plastic selected from the group comprising: polyamide, nylon, polycarbonate, polyethylene, polypropylene, polystyrene, polyurethane, polyvinyl chloride, polyvinylidene chloride, acrylonitrile butadiene styrene, polyetheretherketone, and silicone.

Optionally, the grip comprises polyester. Optionally, the grip comprises Polybutylene Terephthalate.

Optionally, the grip comprises a combination of Polybutylene Terephthalate and glass fibre. Optionally, the grip comprises a combination of Polybutylene Terephthalate and glass fibre in a proportion within the range 0-60% (w/w) glass fibre. Optionally, the grip comprises a combination of Polybutylene Terephthalate and glass fibre in a proportion selected from the group comprising: 10% (w/w) glass fibre, 20% (w/w) glass fibre, 25% (w/w) glass fibre, 30% (w/w) glass fibre, 35% (w/w) glass fibre, 40% (w/w) glass fibre, 50% (w/w) glass fibre, and 60% (w/w) glass fibre.

Optionally, the grip comprises synthetic rubber. Optionally, the grip comprises polyisoprene rubber.

Optionally, the grip comprises natural rubber. Optionally, the grip comprises non-synthetic rubber. Optionally, the grip comprises hevea rubber.

Optionally, the grip comprises metal. Optionally, the grip comprises metal alloy. Optionally, the grip comprises a metal selected from the group comprising: iron, steel, stainless steel, aluminium, and copper. Optionally, the grip comprises high carbon steel.

Optionally, the at least one clamp is displaceable between an open position and a closed position.

Optionally, the at least one clamp comprises a pair of jaws. Optionally, the pair of jaws is displaceable between an open position and a closed position.

Optionally, the pair of jaws defines an opening therebetween.

Optionally, the pair of jaws comprises a first jaw and a second jaw.

Optionally, in the open position, the pair of jaws defines an opening between the first jaw and the second jaw. Optionally, in the open position, the pair of jaws defines an opening between the first jaw and the second jaw towards one end of the pair of jaws.

Optionally, in the closed position, the first jaw abuts the second jaw. Optionally, in the closed position, the first jaw abuts the second jaw towards one end of the pair of jaws.

Optionally, the at least one clamp is biased toward the closed position.

Optionally, the at least one clamp is hingedly arranged.

Optionally, the at least one clamp comprises a clamp hinge. Optionally, the clamp hinge comprises a clamp live hinge.

Optionally, at least one of the jaws pivots about the hinge in moving from the open position to the closed position of the pair of jaws, and vice versa.

Optionally, the at least one guide is displaceable between a wide position and a narrow position.

Optionally, the at least one guide comprises a pair of arms. Optionally, the pair of arms is displaceable between a wide position and a narrow position.

Optionally, the pair of arms defines the aperture therebetween. Optionally, the pair of arms defines the aperture therebetween, towards one end of the pair of arms.

Optionally, the pair of arms comprises a first arm and a second arm.

Optionally, the distance across the aperture between the pair of arms is greater in the wide position than in the narrow position. Optionally, in the narrow position, the first arm abuts the second arm.

Optionally, the at least one guide is biased toward the narrow position.

Optionally, the at least one guide is hingedly arranged.

Optionally, the at least one guide comprises a guide hinge. Optionally, the guide hinge comprises a guide live hinge.

Optionally, at least one of the arms pivots about the guide hinge in moving from the wide position to the narrow position of the pair of arms, and vice versa.

Optionally, the at least one clamp is collocated with the at least one guide. Optionally, the pair of arms is collocated with the pair of jaws. Optionally, the guide hinge is collocated with the clamp hinge. Optionally, the aperture of the guide is collocated with the clamp hinge. Optionally, the opening of the clamp is collocated with the guide hinge.

Optionally, in use, the at least one wire is retained at the at least one clamp in the closed position.

Optionally, in use, the at least one wire is retained coaxially at the at least one clamp in the closed position.

Optionally, in use, the at least one wire is retained at the at least one guide.

Optionally, in use, the at least one wire is retained coaxially at the at least one guide.

Optionally, in use, the at least one wire is retained at the at least one guide in the narrow position.

Optionally, in use, the at least one wire is released from the at least one guide in the wide position.

Optionally, in use, the at least one wire is retained coaxially at the at least one guide in the narrow position.

Optionally, the at least one aperture of the guide comprises a longest dimension, whereby the length of the at least one aperture in the longest dimension is greater than the length of the at least one aperture in substantially any other dimension.

Optionally, the clamp hinge of the at least one clamp is substantially parallel to the longest dimension of the at least one aperture.

Optionally, the guide hinge of the at least one guide is substantially parallel to the longest dimension of the at least one aperture.

Optionally, the at least one guide is channel shaped. Optionally, the at least one guide has a substantially u-shaped cross-section. Optionally, the at least one guide has a substantially penannular cross-section. Optionally, the at least one guide has a substantially slit-tube shaped cross-section.

Optionally, the at least one guide comprises a guide through-hole. Optionally, the at least one guide is substantially tube-shaped.

Optionally, the grip further comprises at least one through-hole. Optionally, the at least one through-hole is each adapted to receive one or more wires.

Optionally, the at least one guide is adapted to receive at least one wire. Optionally, the at least one wire may be any filamentous body. Optionally, the at least one guide is adapted to receive at least one filamentous body selected from the group comprising: lines, wires, tubes, hoses, and/or catheters. Optionally, the at least one wire may be any elongated body.

Optionally, the at least one lip is connected to at least one of the pair of arms. Optionally, the at least one lip is connected to the arm in an arrangement distant from the guide hinge.

Optionally, the at least one lip is integrally formed in at least one of the pair of arms. Optionally, the at least one lip is integrally formed in at least one of the pair of arms at the distal end of the at least one of the pair of arms.

Optionally, the at least one lip has a curve-shaped cross-section. Optionally, the at least one lip has a substantially curvilinear-shaped cross-section. Optionally, the at least one lip has a hook-shaped cross-section.

Optionally, the at least one lip has a curve-shaped cross-section in the plane perpendicular to the longest dimension of the at least one aperture. Optionally, the at least one lip has a substantially curvilinear-shaped cross-section in the plane perpendicular to the longest dimension of the at least one aperture. Optionally, the at least one lip has a hook-shaped cross-section in the plane perpendicular to the longest dimension of the at least one aperture.

Optionally, the grip comprises two guides each adapted to receive one or more wires.

Optionally, the grip further comprises at least one restraining member. Optionally, the at least one restraining member is attached to the inner wall of the at least one guide.

Optionally, the at least one restraining member comprises a resilient material. Optionally, the at least one restraining member comprises a hook-shaped cross-section. Optionally, the at least one restraining member comprises a hook-shaped cross-section in the plane perpendicular to the longest dimension of the at least one aperture.

Optionally, the at least one restraining member comprises a metal material. Optionally, the at least one restraining member comprises a resilient metal material. Optionally, the at least one restraining member comprises steel.

Optionally, the grip further comprises a securing member.

Optionally, the securing member comprises a resilient material. Optionally, the securing member comprises a resilient metal material. Optionally, the securing member comprises steel. Optionally, the securing member comprises high carbon steel.

Optionally, the securing member is adapted to bias the at least one clamp toward the closed position. Optionally, the securing member is connected to the grip on opposite sides of the clamp hinge.

Optionally, the securing member comprises a plate shape. Optionally, the securing member comprises a band shape. Optionally, the securing member comprises a spring shape. Optionally, the securing member comprises a coiled spring shape.

Optionally, the grip may be formed by injection moulding. Optionally, the grip comprises parts that may be formed by injection moulding.

Optionally, there are indentations on the surface of the grip. Optionally, the indentations are arranged in clusters of indentations.

Optionally, there are indentations on opposite sides of the clamp hinge. Optionally, there are clusters of indentations on opposite sides of the clamp hinge.

Optionally, in use the indentations aid grip by increasing friction between the user's contact and the surface of the grip. Optionally, in use the indentations indicate where the user should apply force to the grip in order to move the clamp between the closed position and the open position, and vice versa.

Optionally, there are indentations on opposite sides of the guide hinge. Optionally, there are clusters of indentations on opposite sides of the guide hinge.

Optionally, in use the indentations aid grip by increasing friction between the user's contact and the surface of the grip. Optionally, in use the indentations indicate where the user should apply force to the grip in order to move the guide between the narrow position and the wide position, and vice versa.

Optionally, some or all of the indentations are bulges.

Optionally, the clamp is adapted to reversibly fasten the grip to a flexible surface. Optionally, the clamp is adapted to reversibly fasten the grip to a fabric surface. Optionally, the clamp is adapted to reversibly fasten the grip to a fabric surface selected from the group comprising: clothing material, bed clothing material, a pillowcase, a bedsheet, a blanket, and a duvet cover.

Optionally, there are teeth connected to at least one of the pair of jaws. Optionally, in use the teeth increase friction between the jaw and the surface.

Optionally, the width of the grip in at least one dimension is less than 20 millimetres. Optionally, the width of the grip in at least one dimension is less than a distance selected from the group comprising: 10 millimetres, 20 millimetres, 25 millimetres, 30 millimetres, 35 millimetres, and 40 millimetres.

In the context of this specification, the term live hinge refers to both a single axis hinge formed by a fixed flexible portion of a material, and a live hinge also refers to material that may flex or bend along its length, whereby there is no obvious fixed hinge axis in the material.

### EXAMPLES

### Example 1

The final design consists of three parts in total, a securing member comprising spring steel and two other parts comprising plastic. A considerable development was the implementation of spring steel material; this spring steel material brings functionality together all in one by biasing the grip handles together, biasing the jaws of the clamp toward the closed position of the clamp, and finally biasing the guides(s) for the wires or other filamentous bodies toward the narrow position. The interaction to open the grip is simple by pinching the two sides.

To restrain the wire or other filamentous body in the guide requires the user to move the guide into the wide position and push the wire into the guide, a small arrow is indicated where to do this. The wire can be locked by the restraining member; to do this the user pushes the wire under the restraining member. Due to the resilience of the restraining member, there is a slight snap sensation. The restraining member keeps the wire in place when the guide is moved to the wide position.

The clamp of the grip can open if the wire or other filamentous body is in the lock position, or if the at least one guide is in the narrow position, in case the user needs to move the grip place on the bed sheet but does not need the wire(s) or other filamentous bodies unlocked or released from the guide.

To unlock the wire or other filamentous body the user again moves the guide toward the wide position and pushes the wire or other elongated body into the unlock position.

In total this grip can guide three wires or other filamentous bodies comfortably with the option to lock one wire. A guide on one half of the grip functions to guide wires or other filamentous bodies—the wires or other filamentous bodies can be pushed into this guide easier than the wire lock/unlock guide. A considerable requirement from research is that the grip must live on the nurse call device wire; the wire lock guide of the grip requires more strength to insert a wire into through the aperture, so that it is discouraged from being removed from the driver nurse call device wire. This design feature means that the grip will not become lost in this environment where equipment is moved constantly. There is also a restraining member attached to the inner wall of the wire lock guide.

The restraining member comprises a notch in the wire lock guide of the grip, or alternatively the restraining member comprises a clip attached to the inner wall of the wire lock guide. The restraining member keeps the wire in place when the relevant guide is moved to the wide position.

When the user wants to push a wire into the lock position there is a slight snap sensation. Wires from 3 mm to 5 mm will work with this design as the restraining member comprising spring steel gives adjustability.

### Example 2

This embodiment can lock a wire but also guide and constrain more wires.

The spring or resilience of the plastic material will be relied upon a small amount to keep the lip of the guide aperture closed.

The securing member comprising spring steel implemented in this grip gives strength to the clamp hinge of the plastic material at the base which would otherwise be weak, biasing the clamp towards the closed position.

The locking of the wire is similar to that of clip 1, the wire is pushed into a restraining member comprising a spring steel clip.

Indentations on the sides of the grip aid with grip and signal to the user where to apply force.

### Additional Embodiments

In at least one embodiment, there is provided a grip 10 for guiding one or more wires 16, the grip 10 comprising:
(a) at least one clamp 1 adapted to reversibly fasten the grip 10 to a surface, and
(b) at least one guide 2 adapted to receive the one or more wires 16.

In some embodiments, the guide 2 comprises at least one aperture 6. The grip 10 may comprise at least one lip 14 adjacent to the aperture 6 wherein the at least one lip 14 is formed from resilient material.

In some embodiments, the grip 10 is a unitary grip 10. For example, the grip 10 may be formed as a single continuous part. For example, the grip 10 may be formed from a single material. However, the grip 10 may also be formed from one or more parts. Such a grip 10 may be formed from two or more materials. The materials can be the same or different materials. In some embodiments, the grip 10 is formed from a resilient material, or a single resilient material. Alternatively, the grip 10 is formed from two or more resilient materials. The resilient materials can be the same or different resilient materials. In some embodiments, the grip 10 comprises at least one material selected from the group comprising: plastic, rubber, synthetic resin, glass fibre, and metal. For example, the grip 10 may comprise a plastic selected from the group comprising: polyamide, nylon, polycarbonate, polyethylene, polypropylene, polystyrene, polyurethane, polyvinyl chloride, polyvinylidene chloride, acrylonitrile butadiene styrene, polyetheretherketone, and silicone. In some embodiments, the grip 10 comprises polyester. For example, the grip 10 may comprise Polybutylene Terephthalate. In some embodiments, the grip 10 comprises a combination of Polybutylene Terephthalate and glass fibre. For example, the grip 10 may comprise a combination of Polybutylene Terephthalate and glass fibre in a proportion within the range 0-60% (w/w) glass fibre. For example, the grip 10 may comprise a combination of Polybutylene Terephthalate and glass fibre in a proportion selected from the group comprising: 10% (w/w) glass fibre, 20% (w/w) glass fibre, 25% (w/w) glass fibre, 30% (w/w) glass fibre, 35% (w/w) glass fibre, 40% (w/w) glass fibre, 50% (w/w) glass fibre, and 60% (w/w) glass fibre. In some embodiments, the grip 10 comprises synthetic rubber. For example, the grip 10 may comprise polyisoprene rubber. Alternatively, the grip 10 may comprise natural rubber. For example, the grip 10 may comprise non-synthetic rubber or hevea rubber. In some embodiments, the grip 10 comprises metal. For example, the grip 10 may comprise metal alloy. In such embodiments, the grip 10 may comprise a metal selected from the group comprising: iron, steel, stainless steel, aluminium, and copper. For example, the grip 10 may comprise high carbon steel.

In some embodiments, the at least one clamp 1 is displaceable between an open position and a closed position. For example, the at least one clamp 1 may comprise a pair of jaws 3; in which case the pair of jaws 3 is displaceable between an open position and a closed position. In some embodiments, the pair of jaws 3 defines an opening 5 therebetween. The skilled person would understand that the pair of jaws 3 may comprise a first jaw and a second jaw. For example, in the open position, the pair of jaws 3 may define an opening 5 between the first jaw and the second jaw; the opening 5 may be towards one end of the pair of jaws 3. Meanwhile, in the closed position, the first jaw may abut or contact the second jaw; for example the abutment of contact between the pair of jaws 3 may be towards one end of the pair of jaws 3. In some embodiments, the at least one clamp 1 is biased toward the closed position. The skilled person would understand that the at least one clamp 1 may be hingedly arranged. For example, the at least one clamp 1 may comprise a clamp hinge 7. Alternatively, the at least one clamp 1 may comprise a clamp 1 live hinge. The skilled person would understand that at least one of the jaws may pivot about the hinge in moving from the open position to the closed position of the pair of jaws 3, and vice versa.

In some embodiments, the at least one guide 2 is displaceable between a wide position and a narrow position. For example, the at least one guide 2 may comprise a pair of arms 4; wherein the pair of arms 4 is displaceable between a wide position and a narrow position. In some embodiments, the pair of arms 4 defines the aperture 6 therebetween; optionally the aperture 6 is positioned towards one end of the pair of arms 4. The skilled person would understand that the pair of arms 4 may comprise a first arm and a second arm. In some embodiments, the distance across the aperture 6 between the pair of arms 4 is greater in the wide position than in the narrow position. In some embodiments, in the narrow position, the first arm abuts the second arm. In some embodiments, the at least one guide 2 is biased toward the narrow position. For example, the at least one guide 2 may be hingedly arranged. In such embodiments, the at least one guide 2 may comprises a hinge which will be referred to as a guide hinge 8. The guide hinge 8 may be a live hinge, which will be referred to as a guide 2 live hinge. The skilled person would understand that at least one of the arms may pivot about the guide hinge 8 in moving from the wide position to the narrow position of the pair of arms 4, and vice versa. In some embodiments, the at least one clamp 1 is collocated with the at least one guide 2; for example the pair of arms 4 may be collocated with the pair of jaws 3; furthermore, the guide hinge 8 may be collocated with the clamp hinge 7. In such embodiments, the aperture 6 of the guide 2 may be collocated with the clamp hinge 7; and/or the opening 5 of the clamp 1 may be collocated with the guide hinge 8.

The skilled person would understand that, in use, the at least one wire may be retained at or by the at least one clamp 1 in the closed position; for example the at least one wire may be retained coaxially at or by the at least one clamp 1 in the closed position. Furthermore, the skilled person would understand that, in use, the at least one wire may be retained at or by the at least one guide 2. For example, in use, the at least one wire may be retained coaxially at or by the at least one guide 2. In some embodiments the at least one wire may be retained by the at least one guide 2 only in the narrow position; for example the at least one wire may be released from the at least one guide 2 in the wide position. The skilled person would understand that, in use, the at least one wire may be retained coaxially at or by the at least one guide 2 in the narrow position.

The skilled person would understand that the at least one aperture 6 of the guide 2 comprises a longest dimension, whereby the length of the at least one aperture 6 in the longest dimension is greater than the length of the at least one aperture 6 in substantially any other dimension. In some embodiments, the clamp hinge 7 of the at least one clamp 1 is substantially parallel to the longest dimension of the at least one aperture 6. Alternatively or additionally, the guide hinge 8 of the at least one guide 2 may be substantially parallel to the longest dimension of the at least one aperture 6.

In some embodiments, the at least one guide 2 is channel shaped. For example, the at least one guide 2 may have a substantially u-shaped cross-section, or a substantially penannular cross-section, or a substantially slit-tube shaped cross-section. In some embodiments, the at least one guide 2 comprises a guide through-hole. In such embodiments, the at least one guide 2 may be substantially tube-shaped.

In some embodiments, the grip 10 further comprises at least one through-hole 12; such at least one through-hole 12 may be adapted to receive one or more wires 16.

In some embodiments, the at least one guide 2 is adapted to receive at least one wire. In some embodiments, the at least one wire may be any filamentous body. For example, in some embodiments the at least one guide 2 is adapted to receive at least one filamentous body selected from the group comprising: lines, wires 16, tubes, hoses, and/or catheters. Furthermore, the at least one wire may be replaced by any elongated body; such that in some embodiments, the at least one guide 2 is adapted to receive at least one elongated body.

The at least one lip 14 may be connected to at least one of the pair of arms 4. In such embodiments, the at least one lip 14 may be connected to the arm in an arrangement distant from the guide hinge 8. For example, the at least one lip 14 may be integrally formed in at least one of the pair of arms 4; optionally at the distal end of the at least one of the pair of arms 4. In some embodiments, the at least one lip 14 has a curve-shaped cross-section; optionally a substantially curvilinear-shaped cross-section; optionally a hook-shaped cross-section. The skilled person would understand that, in some embodiments, the at least one lip 14 may have a curve-shaped cross-section, or substantially curvilinear-shaped cross-section, or hook-shaped cross-section, in the plane perpendicular to the longest dimension of the at least one aperture 6.

The grip 10 may comprise two guide 2s each adapted to receive one or more wires 16.

In some embodiments, the grip 10 further comprises at least one restraining member 11; which in some embodiments may be attached to the inner wall of the at least one guide 2. In some embodiments, the at least one restraining member 11 comprises a resilient material. The at least one restraining member 11 may have a hook-shaped cross-section; for example the at least one restraining member 11 may have a hook-shaped cross-section in the plane perpendicular to the longest dimension of the at least one aperture 6. In some embodiments, the at least one restraining member 11 comprises a metal material. The skilled person would understand that the at least one restraining member 11 may comprise a resilient metal material; for example steel, or any other suitable material.

In some embodiments, the grip 10 further comprises a securing member 9. The securing member 9 may in some embodiments comprise a resilient material. The skilled person would understand that the securing member 9 may comprise resilient metal material; for example steel or high carbon steel, or any other suitable material. In some embodiments, the securing member 9 is adapted to bias the at least one clamp 1 toward the closed position. For example, the securing member 9 may be connected to the grip 10 on opposite sides of the clamp hinge 7. In some embodiments, the securing member 9 comprises a plate shape, or a band shape, or a spring shape. For example, in some embodiments the securing member 9 comprises a coiled spring shape.

The skilled person would understand that in at least some embodiments, the grip 10 may be formed by injection moulding. In some such embodiments, the grip 10 comprises parts that may be formed by injection moulding.

In some embodiments, there are indentations on the surface of the grip 10. The indentations could be arranged in clusters of indentations. For example, there may be indentations, or clusters of indentations, on opposite sides of the clamp hinge 7. The skilled person would understand that, in use, the indentations may aid grip 10 by increasing friction between the user's contact and the surface of the grip 10; and/or, in use, the indentations may indicate where the user should apply force to the grip 10 in order to move the clamp 1 between the closed position and the open position, and vice versa. Alternatively or additionally, in use, the indentations may indicate where the user should apply force to the grip 10 in order to move the guide 2 between the narrow position and the wide position, and vice versa. The indentations, or clusters of indentations, might be positioned on opposite sides of the guide hinge 8. The skilled person would understand that the indentations could be replaced by or supplemented with other surface depressions or raised areas. For example, in some embodiments some or all of the indentations are bulges 15.

In some embodiments, the clamp 1 is adapted to reversibly fasten the grip 10 to a flexible surface. For example, the clamp 1 might be adapted to reversibly fasten the grip 10 to a fabric surface. Such a fabric surface would include fabric surfaces selected from the group comprising: clothing material, bed clothing material, a pillowcase, a bedsheet, a blanket, and a duvet cover. In some embodiments, there are teeth 13 connected to at least one of the pair of jaws 3. In use the teeth 13 may increase friction between the jaw and the surface.

In some embodiments, the width of the grip 10 in at least one dimension is less than 20 millimetres. In some embodiments, the width of the grip 10 in at least one dimension is less than a distance selected from the group comprising: 10 millimetres, 20 millimetres, 25 millimetres, 30 millimetres, 35 millimetres, and 40 millimetres.

In at least one embodiment, the grip 10 is a unitary grip 10. The grip 10 is formed as a single continuous part. Furthermore, the grip 10 is formed from a single resilient material. The at least one clamp 1 comprises a clamp 1 live hinge; and the grip 10 comprises one such clamp 1. Said grip 10 is formed by injection moulding. The at least one clamp 1 is collocated with the at least one guide 2, such that the pair of arms 4 is collocated with the pair of jaws 3 and the guide hinge 8 is collocated with the clamp hinge 7. The aperture 6 of the guide 2 is collocated with the clamp hinge 7.

In at least one embodiment, the grip 10 is formed from one or more parts. Furthermore, the grip 10 comprises two or more different materials. The at least one clamp 1 comprises a clamp hinge 7. Said grip 10 further comprises a securing member 9 comprising a resilient metal material. The securing member 9 is adapted to bias the at least one clamp 1 toward the closed position. The securing member 9 is connected to the grip 10 on opposite sides of the clamp hinge 7, and the securing member 9 has a coiled spring shape. There are teeth 13 connected to the at least one jaw; whereby, in use the teeth 13 increase friction between the at least one jaw and the surface. The grip 10 comprises two guide 2s. Each guide 2 comprises a guide 2 live hinge. The two guide 2s are arranged on opposite sides of the clamp hinge 7. There is a lip 14 integrally formed in one of the pair of arms 4 of each of the aperture 6s, in an arrangement such that each lip 14 is at the distant end of the respective arm from the guide 2 live hinge. The lips 14 are formed from resilient material.

In at least one embodiment, the grip 10 is formed from one or more parts. Furthermore, the grip 10 comprises two or more different materials. The at least one clamp 1 comprises a clamp hinge 7. Said grip 10 further comprises a securing member 9 comprising a resilient metal material. The securing member 9 is adapted to bias the at least one clamp 1 toward the closed position. The securing member 9 is connected to the grip 10 on opposite sides of the clamp hinge 7, and the securing member 9 has a plate or band shape. There are teeth 13 connected to the at least one jaw; whereby, in use the teeth 13 increase friction between the at least one jaw and the surface. The grip 10 comprises two guide 2s. Each guide 2 comprises a guide 2 live hinge. The two guide 2s are arranged on opposite sides of the clamp hinge 7. There is a lip 14 integrally formed in one of the pair of arms 4 of each of the aperture 6s, in an arrangement such that each lip 14 is at the distant end of the respective arm from the guide 2 live hinge. The lip 14s are formed from resilient material. The grip 10 further comprises a restraining member 11 attached to the inner wall of one of the guide 2s. The restraining member 11 is made of a resilient material and has a hook-shaped cross-section.

In at least one embodiment, the grip 10 is formed from one or more parts. Furthermore, the grip 10 comprises two or more different materials. The at least one clamp 1 comprises a clamp hinge 7. Said grip 10 further comprises a securing member 9 comprising a resilient metal material. The securing member 9 is adapted to bias the at least one clamp 1 toward the closed position. The securing member 9 is connected to the grip 10 on opposite sides of the clamp hinge 7, and the securing member 9 has a plate or band shape. There are teeth 13 connected to the at least one jaw; whereby, in use the teeth 13 increase friction between the at least one jaw and the surface. The grip 10 comprises two guide 2s. One guide 2 comprises a guide 2 live hinge, while the other guide 2 comprises a guide hinge 8. The two guide 2s are arranged on opposite sides of the clamp hinge 7. There is a lip 14 integrally formed in one of the pair of arms 4 of each of the aperture 6s, in an arrangement such that each lip 14 is at the distant end of the respective arm from the guide hinge 8 (or guide 2 live hinge). The lips 14 are formed from resilient material. The grip 10 further comprises a through-hole 12. The through-hole 12 is each adapted to receive one or more wires 16. The width of the grip 10 in at least one dimension is less than 20 millimetres.

In at least one embodiment, the grip 10 is formed from one or more parts. Furthermore, the grip 10 comprises two or more different materials. The at least one clamp 1 comprises a clamp hinge 7. Said grip 10 further comprises a securing member 9 comprising a resilient metal material. The securing member 9 is adapted to bias the at least one clamp 1 toward the closed position. The securing member 9 is connected to the grip 10 on opposite sides of the clamp hinge 7, and the securing member 9 has a plate or band shape. There are teeth 13 connected to the at least one jaw; whereby, in use the teeth 13 increase friction between the at least one jaw and the surface. The grip 10 comprises two guide 2s. Each guide 2 comprises a guide 2 live hinge. The two guide 2s are arranged on opposite sides of the clamp hinge 7. There is a lip 14 integrally formed in one of the pair of arms 4 of each of the aperture 6s, in an arrangement such that each lip 14 is at the distant end of the respective arm from the guide 2 live hinge. The lips 14 are formed from resilient material. The width of the grip 10 in at least one dimension is less than 20 millimetres.

In at least one embodiment, the grip 10 comprises one clamp 1. The clamp 1 comprises a clamp 1 live hinge. The grip 10 comprises one guide 2. The guide 2 comprises a guide 2 live hinge.

In at least one embodiment, the grip 10 comprises one clamp 1. The clamp 1 comprises a clamp hinge 7. The grip 10 comprises one guide 2. The guide 2 comprises a guide 2 live hinge.

In at least one embodiment, the grip 10 comprises one clamp 1. The clamp 1 comprises a clamp 1 live hinge. The grip 10 comprises one guide 2. The guide 2 comprises a guide hinge 8.

In at least one embodiment, the grip 10 comprises one clamp 1. The clamp 1 comprises a clamp hinge 7. The grip 10 comprises one guide 2. The guide 2 comprises a guide hinge 8.

In at least one embodiment, the grip 10 comprises two clamp 1s. The clamp 1s each comprise a clamp 1 live hinge. The grip 10 comprises one guide 2. The guide 2 comprises a guide 2 live hinge.

In at least one embodiment, the grip 10 comprises two clamp 1s. The clamp 1s each comprise a clamp hinge 7. The grip 10 comprises one guide 2. The guide 2 comprises a guide 2 live hinge.

In at least one embodiment, the grip 10 comprises two clamp 1s. The clamp 1s each comprise a clamp 1 live hinge. The grip 10 comprises one guide 2. The guide 2 comprises a guide hinge 8.

In at least one embodiment, the grip 10 comprises two clamp 1s. The clamp 1s each comprise a clamp hinge 7. The grip 10 comprises one guide 2. The guide 2 comprises a guide hinge 8.

In at least one embodiment, the grip 10 comprises one clamp 1. The clamp 1 comprises a clamp 1 live hinge. The grip 10 comprises two guide 2s. The guide 2s each comprise a guide 2 live hinge.

In at least one embodiment, the grip 10 comprises one clamp 1. The clamp 1 comprises a clamp hinge 7. The grip 10 comprises two guide 2s. The guide 2s each comprise a guide 2 live hinge.

In at least one embodiment, the grip 10 comprises one clamp 1. The clamp 1 comprises a clamp 1 live hinge. The grip 10 comprises two guide 2s. The guide 2s each comprise a guide hinge 8.

In at least one embodiment, the grip 10 comprises one clamp 1. The clamp 1 comprises a clamp hinge 7. The grip 10 comprises two guide 2s. The guide 2s each comprise a guide hinge 8.

1. A grip for guiding at least one wire, the grip comprising:
(a) at least one clamp adapted to reversibly fasten the grip to a surface, and
(b) at least one guide adapted to receive the one or more wires; wherein the guide comprises at least one aperture.

2. The grip of claim 1, wherein the grip comprises at least one lip adjacent to the aperture; wherein the at least one lip is formed from resilient material.

3. The grip of claim 2, wherein the at least one lip has a substantially curvilinear-shaped cross-section.

4. The grip of claim 2, wherein the at least one lip has a hook-shaped cross-section in the plane perpendicular to the longest dimension of the aperture.

5. The grip of claim 1, wherein the at least one clamp is displaceable between an open position and a closed position; wherein the at least one clamp is biased toward the closed position.

6. The grip of claim 1, wherein the at least one clamp comprises a pair of jaws displaceable between an open position and a closed position; wherein at least one of the jaws further comprises teeth.

7. The grip of claim 5, wherein the grip further comprises a securing member adapted to bias the at least one clamp toward the closed position; wherein the securing member is connected to the grip on opposite sides of the clamp hinge; and wherein the securing member comprises a resilient material.

8. The grip of claim 7, wherein the securing member comprises a plate shape.

9. The grip of claim 7, wherein the securing member comprises a spring shape.

10. The grip of claim 1, wherein the at least one guide is channel shaped.

11. The grip of claim 1, wherein the at least one guide is adapted to retain the wire coaxially.

12. The grip of claim 1, wherein the grip comprises two guides, wherein each guide is adapted to receive one or more wires.

13. The grip of claim 1, wherein the grip further comprises at least one restraining member attached to the inner wall of the at least one guide.

14. The grip of claim 13, where the at least one restraining member comprises a resilient material.

15. The grip of claim 13, wherein the at least one restraining member comprises a hook-shaped cross-section.

16. The grip of claim 1, wherein the at least one clamp comprises a clamp hinge, wherein the grip further comprises indentations on the surface of the grip on opposite sides of the clamp hinge.

17. The grip of claim 16, whereby the indentations are arranged to indicate where the user should apply force to the grip in order to move the clamp between the closed position and the open position.

18. The grip of claim 16, wherein some or all of the indentations are bulges.

19. The grip of claim 1, wherein the clamp is adapted to reversibly fasten the grip to a fabric surface.

20. The grip of claim 1, wherein the grip comprises at least one material, wherein the material is plastic, rubber, synthetic resin, glass fibre, or metal.
